(19) United States
(12) Patent Application Publication
(10) Pub. No.: US 2024/0173199 A1
(43) Pub. Date: May 30, 2024

(54) NEGATIVE-PRESSURE MASSAGE DEVICE AND NEGATIVE- PRESSURE CONTROLLER

(71) Applicant: BIBOTING INTERNATIONAL CO., LTD., TAOYUAN CITY (TW)

(72) Inventor: Pei-En LEE, New Taipei City (TW)

(21) Appl. No.: 17/994,280

(22) Filed: Nov. 26, 2022

(51) Int. Cl. A61H 9/00; A61H 23/02; A61H 33/06

(52) U.S. Cl. CPC A61H 9/0057; A61H 23/02; A61H 33/06; A61H 2201/0207; A61H 2201/10; A61H 2201/1207; A61H 2201/5005; A61H 2201/5043; A61N 5/0613

(57) ABSTRACT

A negative-pressure controller controls and adjusts a negative pressure and a rhythm intensity generated by at least one accessory body. The negative-pressure controller includes a control panel, a negative-pressure air hole, and a first output port. The control panel is disposed on one side of the negative-pressure controller, and sets an operation parameter. The negative-pressure air hole is formed on one side of the negative-pressure controller, and provides a suction force from the external to the negative-pressure air hole to adjust the negative pressure. The first output port is formed on one side of the negative-pressure controller, and provides a PWM voltage. The negative-pressure controller adjusts a duty cycle of the PWM voltage based on the operation parameter, and the duty cycle is positively correlated with the rhythm intensity.

### BACKGROUND

### Technical Field

The present disclosure relates to a massage device and a controller, and more particularly to a negative-pressure massage device and a negative-pressure controller.

### Description of Related Art

The statements in this section merely provide background information related to the present disclosure and do not necessarily constitute prior art.

The statements in this section merely provide background information related to the present disclosure and do not necessarily constitute prior art. With the progress and development of the industrial and commercial society, in the mode of advocating speed and efficiency, modern people are always under various pressures. Coupled with abnormal diet, excessive intake of food and little exercise, many diseases related to cardiovascular or other unknown sources have occurred. In order to effectively relieve pressure and improve various functions of the body, related companies have successively developed various negative-pressure massage devices for different purposes to meet the needs of the market.

In general, the cups used in negative-pressure device in the industry will be used with a rhythm box, and the vibration generated by a vibration motor in the rhythm box will enhance blood circulation. In the known technology, a vibration intensity adjustment knob is disposed on each rhythm box, and a fixed DC power to all connected rhythm boxes is provided through a multi-connector power line. Therefore, when the vibration intensity needs to be adjusted, the knobs must be adjusted one by one on each rhythm box. Only after the device counts down for 30 minutes, will the DC power be cut off once so that all rhythm boxes will stop operating at the same time. Therefore, each rhythm box must individually adjust the knob to change the vibration mode, thereby causing inconvenience in use.

Accordingly, the present disclosure designs a negative-pressure massage device and a negative-pressure controller to supply power to an accessory body so that the accessory body can directly accept the current input voltage/current, without connecting to the system separately for synchronous control.

### SUMMARY

In order to solve the above-mentioned problem, the present disclosure provides a negative-pressure massage device. The negative-pressure massage device includes at least one accessory body and a negative-pressure controller. The at least one accessory body includes a negative-pressure accessory and a rhythm box. The negative-pressure accessory includes an air hole. The rhythm box is disposed and contacted on one side of the negative-pressure accessory. The rhythm box includes a vibration motor, and the vibration motor drives the negative-pressure accessory to move accordingly. The negative-pressure controller is separately disposed from the at least one accessory body, and the negative-pressure controller includes a control panel, a negative-pressure air hole, and a first output port. The control panel is disposed on one side of the negative-pressure controller, and sets an operation parameter. The negative-pressure air hole is formed on one side of the negative-pressure controller, and communicates with the air hole through an external air pipe to provide a suction force from the external air pipe to the negative-pressure air hole, and forms a negative pressure from the air hole to the external air pipe. The first output port is formed on one side of the negative-pressure controller, electrically connected to the rhythm box through a power line, and provides a PWM voltage to the vibration motor through the power line. The negative-pressure controller adjusts a duty cycle of the PWM voltage based on the operation parameter to change a rhythm intensity of the vibration motor.

In order to solve the above-mentioned problem, the present disclosure provides a negative-pressure controller. The negative-pressure controller controls and adjusts a negative pressure and a rhythm intensity generated by at least one accessory body. The negative-pressure controller includes a control panel, a negative-pressure air hole, and a first output port. The control panel is disposed on one side of the negative-pressure controller, and sets an operation parameter. The negative-pressure air hole is formed on one side of the negative-pressure controller, and provides a suction force from the external to the negative-pressure air hole. The first output port is formed on one side of the negative-pressure controller, and provides a PWM voltage. The negative-pressure controller adjusts a duty cycle of the PWM voltage based on the operation parameter, and the duty cycle is positively correlated with the rhythm intensity. The suction force is positively correlated with the negative pressure.

The main purpose and effect of the present disclosure are the negative-pressure massage device developed by the present disclosure is dedicated to supply power to the connected accessory body (especially the rhythm box). The first output port of the negative-pressure controller provides PWM voltage for regulation, and then makes the vibration motor speed up or down so that the vibration intensity is the corresponding intensity. On the other hand, the circuit board of the accessory body directly receives the current input voltage/current (that is, the PWM voltage), and does not need to be separately connected to the system for synchronous control, which simplifies the manufacturing cost and control manner of the accessory body.

It is to be understood that both the foregoing general description and the following detailed description are exemplary, and are intended to provide further explanation of the present disclosure as claimed. Other advantages and features of the present disclosure will be apparent from the following description, drawings and claims.

### BRIEF DESCRIPTION OF DRAWINGS

The present disclosure can be more fully understood by reading the following detailed description of the embodiment, with reference made to the accompanying drawing as follows:

FIG. 1A is a schematic appearance view of a negative-pressure massage device according to the present disclosure.

FIG. 1B is a schematic diagram of the correlation between the duty cycle and the rhythm intensity of the PWM voltage according to the present disclosure.

FIG. 2A is a schematic diagram of component configuration from a first perspective of internal circuits of a negative-pressure controller according to the present disclosure.

FIG. 2B is a schematic diagram of component configuration from a second perspective of internal circuits of the negative-pressure controller according to the present disclosure.

FIG. 3 is a schematic diagram of component configuration of an accessory body according to the present disclosure.

### DETAILED DESCRIPTION

Reference will now be made to the drawing figures to describe the present disclosure in detail. It will be understood that the drawing figures and exemplified embodiments of present disclosure are not limited to the details thereof.

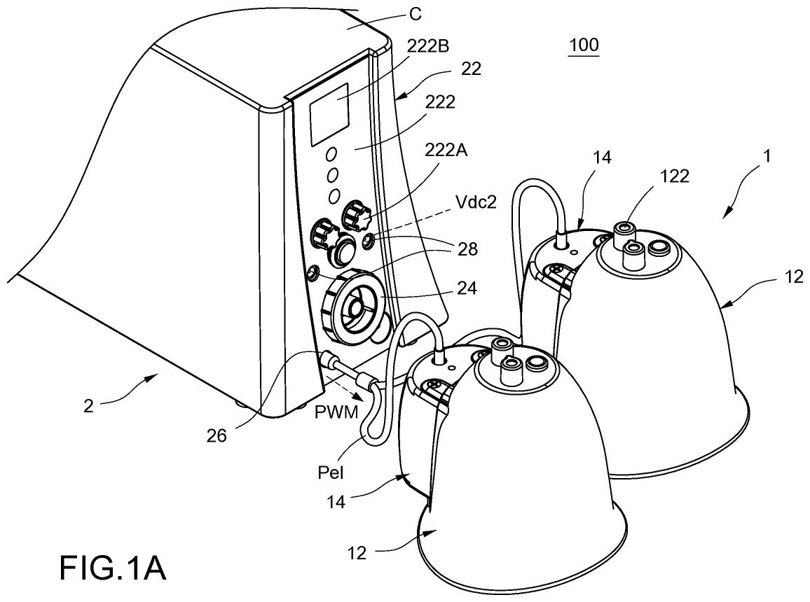

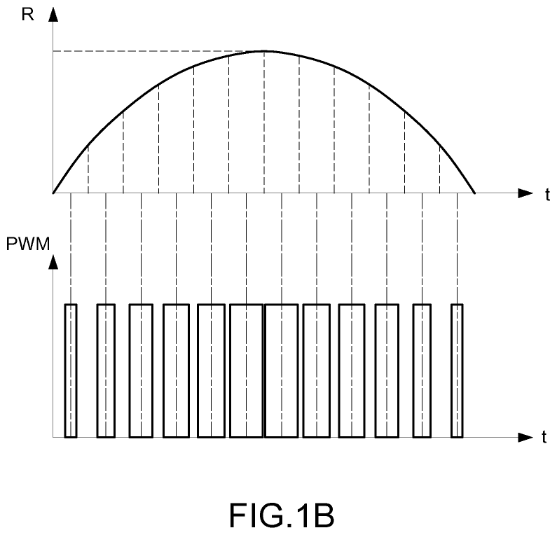

Please refer to FIG. 1A, which shows a schematic appearance view of a negative-pressure massage device according to the present disclosure. The negative-pressure massage device 100 mainly sucks/releases specific parts of the human body for uninterrupted massage so as to effectively relieve muscle tension and fatigue. At the same time, additional accessories (such as phototherapy, heat therapy, etc.) may also be used to increase the effect of relieving muscle tension and fatigue. The negative-pressure massage device 100 includes at least one accessory body 1 and a negative-pressure controller 2, and the negative-pressure controller 2 is separately disposed from the at least one accessory body 1. In particular, the negative-pressure controller 2 is connected to the at least one accessory body 1 through an external circuit connection so as long as the accessory body 1 can be controlled by the negative-pressure controller 2, it can be operated through the external circuit connection. In addition, FIG. 1A is an example of two accessory bodies 1 collocated with the negative-pressure controller 2, but it is not limited thereto, and the number of the accessory bodies 1 may be increased or decreased according to the needs of users.

The accessory body 1 includes a negative-pressure accessory 12 and a rhythm box 14. The negative-pressure accessory 12 includes an air hole 122, and the air hole 122 is used to allow air to enter/exit the negative-pressure accessory 12 so that when the negative-pressure accessory 12 is attached to a specific part of the human body, pressure changes will occur. The rhythm box 14 is disposed and contacted on one side of the negative-pressure accessory 12. A vibration motor is accommodated in an accommodating space inside the rhythm box 14, and the vibration motor drives the negative-pressure accessory 12 to move accordingly. Specifically, when the speed of the vibration motor is increased, the vibration intensity will be intensified, otherwise the vibration intensity of the vibration motor will be weakened. The negative-pressure accessory 12 is affected by the intensified/weakened vibration intensity of the vibration motor to produce a rhythmic effect.

The negative-pressure controller 2 includes a control panel 22, a negative-pressure air hole 24, and a first output port 26. The control panel 22 is disposed on one side of the negative-pressure controller 2. The negative-pressure air hole 24 is formed on one side of the negative-pressure controller 2, and may be on the same side as the control panel 22 or on different sides. When the negative-pressure air hole 24 and the control panel 22 are on the same side, the negative-pressure air hole 24 may be integrated on the control panel 22. The negative-pressure air hole 24 communicates with the air hole 122 through an external air pipe (not shown) so as to provide a suction force from the external air pipe to the negative-pressure air hole 24 and form a negative pressure from the accessory body 1 and the air hole 122 to the external air pipe. Relatively, when the negative pressure of the accessory body 1 must be released, the negative-pressure controller 2 introduces air into the negative-pressure air hole 24 to release the negative pressure of the accessory body 1 by releasing the suction force of the negative-pressure air hole 24. The first output port 26 is formed on one side of the negative-pressure controller 2, and may be on the same side as the control panel 22 or on different sides. The first output port 26 may be integrated on the control panel 22. The first output port 26 is electrically connected to the vibration motor inside the rhythm box 14 through a power linePel, and provides a PWM (pulse-width modulation) voltage PWM to the vibration motor through the power line Pe1.

In general, when the PWM voltage PWM is, for example, but not limited to, at a high level, the vibration motor increases its rotation speed to produce an effect of intensified vibration. Conversely, when the PWM voltage PWM is at a low level, the vibration motor decreases its rotation speed to produce an effect of weakened vibration. Therefore, by controlling the vibration intensity of the vibration motor through the PWM voltage PWM can cause the negative-pressure accessory 12 to produce a rhythmic effect.

Furthermore, the control panel 22 may be used to set operation parameters, such as but not limited to massage time, rhythm intensity (for example, but not limited to, it may include constant rhythm intensity, decreasing rhythm intensity or increasing rhythm intensity), frequency of releasing the suction force, etc. Therefore, the negative-pressure controller 2 can adjust the duty cycle of the PWM voltage PWM, the suction force of the negative-pressure air hole 24, the frequency of releasing the suction force based on the operation parameters. Please refer to FIG. 1B, which shows a schematic diagram of the correlation between the duty cycle and the rhythm intensity of the PWM voltage according to the present disclosure. Since the rhythm intensity is positively correlated with the duty ratio (i.e., the on-time width) of the PWM voltage PWM, the negative-pressure controller 2 may adjust the duty ratio of the PWM voltage PWM based on the operation parameters to adjust the rhythm intensity R of the vibration motor. When the negative-pressure controller 2 provides the PWM voltage PWM with a smaller duty ratio, the rhythm intensity R of the vibration motor is smaller. Conversely, when the negative-pressure controller 2 provides the PWM voltage PWM with a larger duty ratio, the rhythm intensity R of the vibration motor is larger.

Furthermore, the negative-pressure controller 2 acquires the required waveform equivalently by modulating the width of a series of pulses, and then uses the high-resolution counter to calculate the duty cycle of the square wave to encode the analog signal alignment bits, and the voltage/current that activates the accessory body 1 by an on and/or off control so that the repeated pulse sequence to simulate the rhythm.

Please refer to FIG. 1A again, the negative-pressure controller 2 further includes a casing C and a second output port 28, and the control panel 22 includes an operation panel 222. The casing C forms an accommodating space inside the casing C (not shown) for accommodating multiple components constituting the negative-pressure controller 2, which will be further described later. In particular, the casing C and the control panel 22 may commonly form the accommodating space inside the negative-pressure controller 2. Alternatively, the casing C alone forms the accommodating space, and then the control panel 22 is installed on the casing C through an opening of the casing C. The second output port 28 is formed on one side of the negative-pressure controller 2, and may be on the same side as the control panel 22 or on different sides. The second output port 28 may be integrated on the control panel 22. The second output port 28 is used to output a second DC voltage Vdc2 with a fixed voltage value, and the second DC voltage Vdc2 may be electrically connected to the accessory body 1 through another power line (not shown) to supply power to the accessory body 1.

Specifically, in addition to the vibration motor of the rhythm box 14 of the accessory body 1 needs to be powered, the accessory body 1 may also include additional accessories, which must be powered by an additional power supply with a fixed voltage value in order to operate smoothly. Therefore, the second DC voltage Vdc2 (such as but not limited to 12 volts) with the fixed voltage value may be provided through the second output port 28 to supply power to the additional accessories in the accessory body 1. In addition, if the vibration motor of the rhythm box 14 continues to be powered by a high-level voltage, the vibration motor will continuously vibrate at the maximum vibration intensity. Therefore, if the vibration motor of the vibration box 14 is changed to be powered by the second DC voltage Vdc2 with the fixed voltage value through the second output port 28, the vibration intensity R of the vibration motor can be controlled to the maximum vibration value (i.e., continuous vibration).

The operation panel 222 includes an operation interface 222A and a display interface 222B. The operation interface 222A is, for example but not limited to, a knob, a touch screen, a switch, or other components that can be used to set operation parameters. The display interface 222B may be, for example but not limited to, a liquid crystal display, an LED display, and the like with an intuitive display screen so as to correspondingly display the operation parameters set by the user.

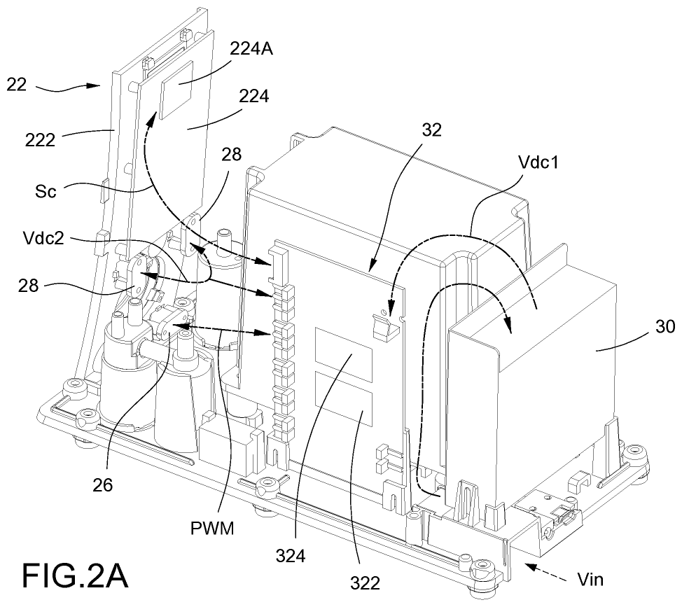

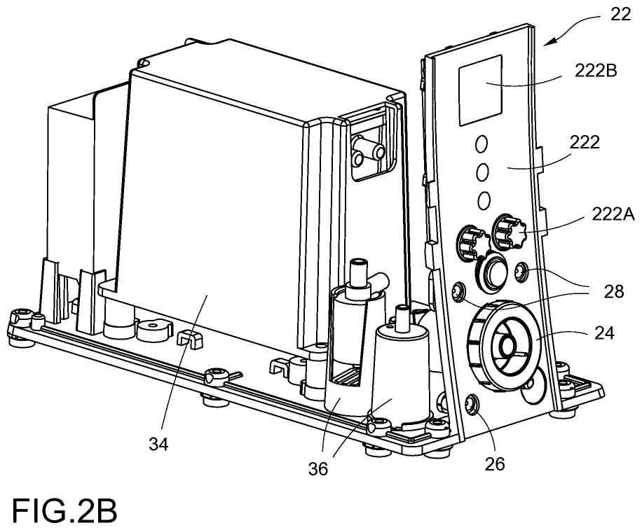

Please refer to FIG. 2A, which shows a schematic diagram of component configuration from a first perspective of internal circuits of a negative-pressure controller according to the present disclosure, and also refer to FIG. 1A and FIG. 1B. The negative-pressure controller 2 is accommodated in the accommodating space inside the casing C, and the negative-pressure controller 2 includes a power supply 30 and a driver board 32, and the control panel 22 further includes a control board 224. The power supply 30 is accommodated in the accommodating space, and converts an input voltage Vin into a first DC voltage Vdc. In one embodiment, the power supply 30 may be an AC/DC converter, and may be a power factor corrector (PFC). The driver board 32 is accommodated in the accommodating space, and is electrically connected to the control panel 22, the first output port 26, and the power supply 30. The power supply 30 provides the first DC voltage Vdc to supply power to the driver board 32, and the driver board 32 drives or supplies power to various components inside the negative-pressure controller 2 based on the control of the control panel 22.

Specifically, the control board 224 is electrically connected to the operation panel 222 and the driver board 32, and the control board 224 provides a control signal Sc corresponding to the operation parameters to the driver board 32 so as to control the driver board 32 through the control signal Sc to drive or supply power to various components inside the negative-pressure controller 2. In particular, a main chip 224A on the control board 224 is responsible for processing the instructions (i.e., operation parameters) sent by the APP or other external controllers, and according to these received instructions, the first output port 26 connected to the same group on the driver board 32 may change the specific power supply mode at the same time.

The driver board 32 includes a first conversion circuit 322 and a second conversion circuit 324. The first conversion circuit 322 is electrically connected to the first output port 26, and the second conversion circuit 324 is electrically connected to the second output port 28. The driver board 32 controls the first conversion circuit 322 to convert the first DC voltage Vdc1 into the PWM voltage PWM, and provide the PWM voltage PWM to the first output port 26. The driver board 32 controls the first conversion circuit 322 to adjust the duty cycle of the PWM voltage PWM based on the control signal Sc (corresponding to the operation parameters). The driver board 32 controls the second conversion circuit 324 to convert the first DC voltage Vdc1 into the second DC voltage Vdc2 with the fixed voltage value so as to provide the second DC voltage Vdc2 with the fixed voltage value to the second output port 28.

In one embodiment, the first conversion circuit 322 and the second conversion circuit 324 are inverters, also may be called AC/DC converters. The driver board 32 controls the switches in the second conversion circuit 324 to be constantly turned on to control the second conversion circuit 324 to convert the first DC voltage Vdc1 into the second DC voltage Vdc2 with the fixed voltage value. Alternatively, a capacitor is used for energy storage to filter the PWM voltage PWM outputted by the second conversion circuit 324 into the second DC voltage Vdc2 with the fixed voltage value. Specifically, since multiple conversion circuits with the same circuit structure are used on the same circuit board (i.e., the driver board 32), the conversion circuit only needs to be designed once, and the selection of components for the conversion circuit is relatively simple. Therefore, the first conversion circuit 322 and the second conversion circuit 324 are designed as the same type of converter, which can simplify the circuit design and reduce the circuit cost.

Please refer to FIG. 2B, which shows a schematic diagram of component configuration from a second perspective of internal circuits of the negative-pressure controller according to the present disclosure, and also refer to FIG. 1A to FIG. 2A. The negative-pressure controller further a negative-pressure generation component 34 and a pressure-releasing component 36. The negative-pressure generation component 34 is electrically connected to the driver board 32, and communicates with the negative-pressure air hole 24 through an internal air pipe (not shown) to provide the suction force. The negative-pressure generation component 34 includes a vacuum pump and a driver circuit of driving the vacuum pump. The driver board 32 is electrically connected to the driver circuit, and controls driver circuit to drive the vacuum pump to adjust the suction force based on the control signal Sc (corresponding to the operation parameters). The vacuum pump is driven by the driver circuit for vacuum suction, and the negative-pressure air hole 24 provides the suction force through the internal air pipe. The pressure-releasing component 36 is electrically connected to the driver board 32, and releases the suction force by communicating the internal air pipe (not shown) to the negative-pressure air hole 24. In one embodiment, the pressure-releasing component 36 is an electromagnetic valve. The driver board 32 controls the pressure-releasing component 36 to release the suction force provided by the negative pressure air hole 24 by introducing air to generate pressure releasing during the negative-pressure process based on the control signal Sc (corresponding to the operation parameters). The driver board 32 controls the pressure-releasing component 36 to adjust a frequency of releasing the suction force based on the control signal Sc (corresponding to the operation parameters) so as to achieve intermittent suction and releasing.

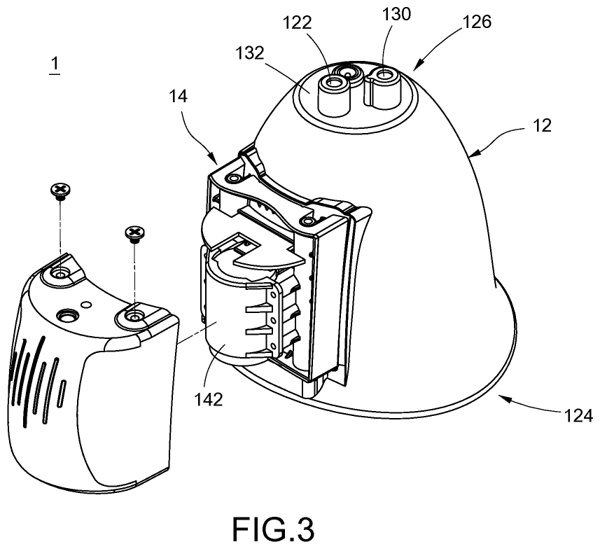

Please refer to FIG. 3, which shows a schematic diagram of component configuration of an accessory body according to the present disclosure, and also refer to FIG. 1A to FIG. 2B. In one embodiment, the accessory body 1 includes a negative-pressure accessory 12 and a rhythm box 14. A vibration motor 142 is installed/disposed inside the rhythm box 14. The negative-pressure accessory 12 is a cup (the following will be represented by cup 12). One end of the cup 12 forms a cup mouth 124 and the other end forms a cup bottom 126 away from the cup mouth 124. The air hole 122 is communicated with the cup bottom 126 so that air enters/exits the cup 12 through the air hole 122. Therefore, when the negative-pressure air hole 24 communicates with the air hole 122 through the external air pipe, and the cup 12 is covered on a specific part of the human body, the air in the cup 12 is sucked out by the suction force through the negative-pressure air hole 24 so that the accommodating space in the cup 12 can produce a negative pressure.

Please refer to FIG. 3 again, the cup 12 further includes an air-releasing valve 130 and an additional component 132. The air-releasing valve 130 is disposed on the cup bottom 126, and manually releases the negative pressure. Specifically, since the negative-pressure controller 2 operates the air suction/releasing operation based on the operation parameters, if it is urgently necessary to terminate the air suction/releasing operation, the user can manually operate the air-releasing valve 130 to release the negative pressure. The additional component 132 is disposed on the cup bottom 126, and receives the second DC voltage Vdc2 with the fixed voltage value to operate. Specifically, the additional component 132 provides an additional function of the cup 12, and the additional function depends on the capabilities of the additional component 132. For example, but not limited to, the additional component 132 may be a phototherapy module with LED lights, which is used to irradiate the direction of the cup mouth 124 to provide the effect of phototherapy. Alternatively, the additional component 132 may be a thermotherapy module with a heating body, which is used to generate heat to provide the effect of thermotherapy. Alternatively, the additional component 132 may be a steam module with solid/liquid atomization to generate mist into the cup 12 to provide the effect of steam therapy. Alternatively, the additional component 132 may be a negative ion emitting module that provides/emits negative ions to provide/emit negative ions that are beneficial to the human body in the direction of the cup 12, and has a good regulating effect on the high-level center of the autonomic nervous system. Alternatively, a combination of the above modules. Therefore, at least one of light therapy, heat therapy, steam therapy, and negative ions can be provided to specific parts of the human body. In one embodiment, the additional component 132 is not limited to the above examples. For example, any additional component 132 that can provide additional functions of relieving muscle tension and fatigue in addition to the suction/releasing massage function should be included in the scope of this embodiment.

Accordingly, the negative-pressure massage device 100 developed by the present disclosure is dedicated to supply power to the connected accessory body 1 (especially the rhythm box 14). It is known that most of the accessory body 1 only needs the ordinary second output port 28 to output a stable 12-volt voltage for operation. However, in order to provide a variety of vibration modes for the rhythm box 14 (such as a round-trip cycle in which the vibration body feels gradually changing from strong to weak, or directly switch between strong, medium, and weak), the present disclosure abandons the manner of synchronously controlling remote connections one by one from the accessories. Instead, the first output port 26 of the negative-pressure controller 2 provides PWM voltage PWM for regulation, and then makes the vibration motor 142 speed up or down so that the vibration intensity is the corresponding intensity. On the other hand, the circuit board of the accessory body 1 directly receives the current input voltage/current (that is, the PWM voltage PWM), and does not need to be separately connected to the system for synchronous control, which simplifies the manufacturing cost and control manner of the accessory body 1.

Although the present disclosure has been described with reference to the preferred embodiment thereof, it will be understood that the present disclosure is not limited to the details thereof. Various substitutions and modifications have been suggested in the foregoing description, and others will occur to those of ordinary skill in the art. Therefore, all such substitutions and modifications are intended to be embraced within the scope of the present disclosure as defined in the appended claims.

What is claimed is:

1. A negative-pressure massage device, comprising:
at least one accessory body, comprising:
a negative-pressure accessory, comprising an air hole, and
a rhythm box, disposed and contacted on one side of the negative-pressure accessory;
the rhythm box comprising a vibration motor, and the vibration motor configured to drive the negative-pressure accessory to move accordingly, and
a negative-pressure controller, separately disposed from the at least one accessory body, and the negative-pressure controller comprising:
a control panel, disposed on one side of the negative-pressure controller, and configured to set an operation parameter,
a negative-pressure air hole, formed on one side of the negative-pressure controller, and configured to communicate with the air hole through an external air pipe to provide a suction force from the external air pipe to the negative-pressure air hole, and form a negative pressure from the air hole to the external air pipe, and
a first output port, formed on one side of the negative-pressure controller, electrically connected to the rhythm box through a power line, and configured to provide a PWM voltage to the vibration motor through the power line,
wherein the negative-pressure controller is configured to adjust a duty cycle of the PWM voltage based on the operation parameter to change a rhythm intensity of the vibration motor.

2. The negative-pressure massage device as claimed in claim 1, wherein the duty cycle is positively correlated with the rhythm intensity.

3. The negative-pressure massage device as claimed in claim 1, wherein the negative-pressure controller further comprises:
a casing, forming an accommodating space,
a power supply, accommodated in the accommodating space, and configured to convert an input voltage into a first DC voltage,
a driver board, accommodated in the accommodating space, and electrically connected to the control panel, the first output port, and the power supply; the driver board comprising a first conversion circuit and a second conversion circuit, and the first conversion circuit electrically connected to the first output port, and
a second output port, formed on one side of the negative-pressure controller, and electrically connected to the second conversion circuit,
wherein the driver board is configured to control the first conversion circuit to convert the first DC voltage into the PWM voltage, and control the first conversion circuit to adjust the duty cycle based on the operation parameter, and
wherein the driver board is configured to control the second conversion circuit to convert the first DC voltage into a second DC voltage for supplying power to the at least one accessory body, or control the rhythm intensity of the vibration motor to be the maximum value.

4. The negative-pressure massage device as claimed in claim 3, wherein the negative-pressure controller further comprises:
a negative-pressure generation component, electrically connected to the driver board, and configured to communicate with the negative-pressure air hole through an internal air pipe to provide the suction force, and
a pressure-releasing component, electrically connected to the driver board, and configured to release the suction force by communicating the internal air pipe with the negative-pressure air hole,
wherein the driver board is configured to adjust the suction force based on the operation parameter, and control the pressure-releasing component to adjust a frequency of releasing the suction force based on the operation parameter.

5. The negative-pressure massage device as claimed in claim 3, wherein the control panel comprises:
an operation panel, comprising:
an operation interface, configured to set the operation parameter, and
a display interface, configured to display the operation parameter, and
a control board, electrically connected to the operation panel and the driver board, and the control board configured to provide a control signal corresponding to the operation parameter to the driver board.

6. The negative-pressure massage device as claimed in claim 3, wherein the negative-pressure accessory is a cup; one end of the cup forms a cup mouth and the other end forms a cup bottom away from the cup mouth, and the air hole is communicated with the cup bottom.

7. The negative-pressure massage device as claimed in claim 6, wherein the cup further comprises:
an air-releasing valve, disposed on the cup bottom, and configured to manually release the negative pressure, and
an additional component, disposed on the cup bottom, and configured to receive the second DC voltage to operate,
wherein the additional component provides an additional function of the cup, and the additional function comprises at least one of phototherapy, heat therapy, steam therapy, and negative ions.

8. A negative-pressure controller, configured to control and adjust a negative pressure and a rhythm intensity generated by at least one accessory body, the negative-pressure controller comprising:
a control panel, disposed on one side of the negative-pressure controller, and configured to set an operation parameter,
a negative-pressure air hole, formed on one side of the negative-pressure controller, and configured to provide a suction force from the external to the negative-pressure air hole, and
a first output port, formed on one side of the negative-pressure controller, and configured to provide a PWM voltage,
wherein the negative-pressure controller is configured to adjust a duty cycle of the PWM voltage based on the operation parameter, and the duty cycle is positively correlated with the rhythm intensity, and
wherein the suction force is positively correlated with the negative pressure.

9. The negative-pressure controller as claimed in claim 8, further comprising:
a casing, forming an accommodating space,
a power supply, accommodated in the accommodating space, and configured to convert an input voltage into a first DC voltage,
a driver board, accommodated in the accommodating space, and electrically connected to the control panel, the first output port, and the power supply; the driver board comprising a first conversion circuit and a second conversion circuit, and the first conversion circuit electrically connected to the first output port, and
a second output port, formed on one side of the negative-pressure controller, and electrically connected to the second conversion circuit,
wherein the driver board is configured to control the first conversion circuit to convert the first DC voltage into the PWM voltage, and control the first conversion circuit to adjust the duty cycle based on the operation parameter, and
wherein the driver board is configured to control the second conversion circuit to convert the first DC voltage into a second DC voltage for supplying power to the at least one accessory body.

10. The negative-pressure controller as claimed in claim 9, wherein the first conversion circuit and the second conversion circuit are inverters.

11. The negative-pressure controller as claimed in claim 9, further comprising:
a negative-pressure generation component, electrically connected to the driver board, and configured to communicate with the negative-pressure air hole through an internal air pipe to provide the suction force, and
a pressure-releasing component, electrically connected to the driver board, and configured to release the suction force by communicating the internal air pipe with the negative-pressure air hole,
wherein the driver board is configured to adjust the suction force based on the operation parameter, and control the pressure-releasing component to adjust a frequency of releasing the suction force based on the operation parameter.

12. The negative-pressure controller as claimed in claim 11, wherein the negative-pressure generation component is a vacuum pump, and the pressure-releasing component is an electromagnetic valve.

13. The negative-pressure controller as claimed in claim 9, wherein the control panel comprises:
an operation panel, comprising:
an operation interface, configured to set the operation parameter, and
a display interface, configured to display the operation parameter, and
a control board, electrically connected to the operation panel and the driver board, and the control board configured to provide a control signal corresponding to the operation parameter to the driver board.
